(19) United States
(12) Patent Application Publication
(10) Pub. No.: US 2005/0103880 A1
(43) Pub. Date: May 19, 2005

(54) Scented air filter

(76) Inventor: Marion Taite, Fort Worth, TX (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 10/985,405

(22) Filed: Nov. 11, 2004

Related U.S. Application Data
(60) Provisional application No. 60/519,719, filed on Nov. 13, 2003.

(51) Int. Cl. A61L 9/04

(52) U.S. Cl. 239/57; 239/56; 239/55; 239/34

(57) ABSTRACT

An air filter assembly includes a perimeter frame supporting an air filtering medium. The perimeter frame defines a cavity holding scented material, and defines apertures that face the air filtering medium, but that are not directly subject to flow-through of air. A cover or tape is adjustable to selectively partially uncover the apertures, thus providing control of the scenting process. By this arrangement, air is scented but the scented material is longer-lasting since it is drawn out of the cavity rather than being blown out or evaporated by high flow of air. Further, the scenting process is controllable by selectively removing the cover or tape, or by selectively positioning of the cover. In some versions, the scented material is replaceable. In another version, the arrangement is attachable to a conventional filter assembly.

### CROSS-REFERENCE TO RELATED APPLICATIONS

This application claims the benefit under 35 U.S.C. § 119(e) of provisional application Serial No. 60/519,719 filed on Nov. 13, 2003 entitled SCENTED AIR FILTER.

### BACKGROUND

The present invention relates to scented air filters.

Consumers enjoy air that smells fresh. In fact, prior art includes air filters having scent applied or attached to air filters for forced air furnaces, where the scent material is attached to or imbedded in the flow-through filter material in various ways. A problem is that in known scented filters, the scent lasts a very short time, since the flow of heated air quickly evaporates the scent material. A relatively high initial concentration of scent is soon followed by ineffective low concentration levels. Further, the amount of scent released is not controllable, and the emission of scent cannot be varied to match the desires of the consumer, nor to meet the needs of a particular heating system being treated with air-freshening scent. Also, known scented air filters are typically not rechargeable. Nor are they configured for retrofit attachment, nor to facilitate assembly. Sometimes, they are not configured for easy attachment to conventionally-configured air filters.

Thus, a system having the aforementioned advantages and solving the aforementioned problems is desired.

### SUMMARY OF THE PRESENT INVENTION

In one aspect of the present invention, an air filter assembly includes a perimeter frame defining an opening adapted to permit flow of air being filtered. The perimeter frame includes a scent-holding member defining a cavity having laterally facing apertures that face the opening but that are located out of direct flow of the air. The assembly further includes a filtering medium adapted to filter the air and that is supported by the perimeter frame and positioned in the opening, and scented material located within the cavity of the scent-holding member for slow passage through the laterally-facing apertures into the flow of air.

In another aspect of the present invention, an air filter assembly includes a perimeter frame defining an opening adapted to permit flow of air being filtered, the perimeter frame including a scent-holding member defining a longitudinally-extending cavity within the scent-holding member, the scent-holding member having apertures that face the opening but that are located out of direct flow of the air and further having an open end. A filtering medium adapted to filter the air is provided and is supported by the perimeter frame and positioned in the opening. Scented material is provided that is adapted to pass through the open end into the cavity of the scent-holding member, and to be retained therein.

In another aspect of the present invention, an air filter assembly includes a perimeter frame defining an opening adapted to permit flow of air being filtered and defining a cavity adjacent the opening and within the cavity. The perimeter frame defines a plurality of apertures facing the opening and includes a strip of material that covers the apertures, the strip being movable to uncover a desired number of the apertures. A filtering medium adapted to filter the air is supported by the perimeter frame and positioned in the opening. Scented material is positioned in the cavity of the scent-holding member. In one form, the strip forms a rigid sliding cover adapted to selectively cover the apertures.

These and other aspects, objects, and features of the present invention will be understood and appreciated by those skilled in the art upon studying the following specification, claims, and appended drawings.

### BRIEF DESCRIPTION OF DRAWINGS

FIG. 1 is a perspective view of an air filter assembly embodying the present invention;

FIGS. 2-3 are perspective views of the scent-holding member in FIG. 1, FIG. 2 showing the tape concealing the apertures in the scent-holding member, and FIG. 3 showing the tape uncovering the apertures;

FIG. 4 is an exploded perspective view of the scent-holding member of FIG. 2;

FIG. 5 is a front view of a first modified air filter assembly;

FIG. 6 is a partial front view of the air filter assembly of FIG. 5, with material being broken away to show the scent-filled cavity in the scent-holding member;

FIG. 7 is a perspective view of the long side section shown in FIG. 6, including showing its cover partially detached from the long side section;

FIG. 8 is a cross-sectional view of the long side section of FIG. 7, wherein scented material is emanating from an internal cavity in vapor form;

FIG. 9 is a partial front view of a second modified air filter assembly having a side section adapted to receive a T-shaped scent-holding member;

FIG. 10 is a partial exploded perspective view of the air filter assembly shown FIG. 9;

FIG. 11 is a partial perspective view of the T-shaped scent-holding member in FIG. 10 wherein a cover or tape has been partially removed to reveal apertures in the T-shaped scent-holding member;

FIG. 12 is a cross-sectional view of the T-shaped scent-holding member of FIG. 11;

FIG. 13 is a partial cross-sectional view of a third modified air filter assembly having a scent-holding member with apertures in an internal cavity that allow vaporization of a liquid or gel scented material to exit the apertures and flow into the air filter assembly;

FIG. 14 is a partial exploded cross-sectional view of the scent-holding member of FIG. 13, including scented material inside an internal cavity in the scent-holding member, the scent-holding member including attachment flanges bent away and prior to assembly onto a conventionally constructed forced air furnace filter;

FIG. 15 is a front view of the air filter assembly of FIG. 13; and

FIG. 16 is a partial perspective view of a bar of solid scented material with tape attached and that is configured to fit into the cavity of the scent-holding member in FIG. 13.

### DETAILED DESCRIPTION OF PREFERRED EMBODIMENTS

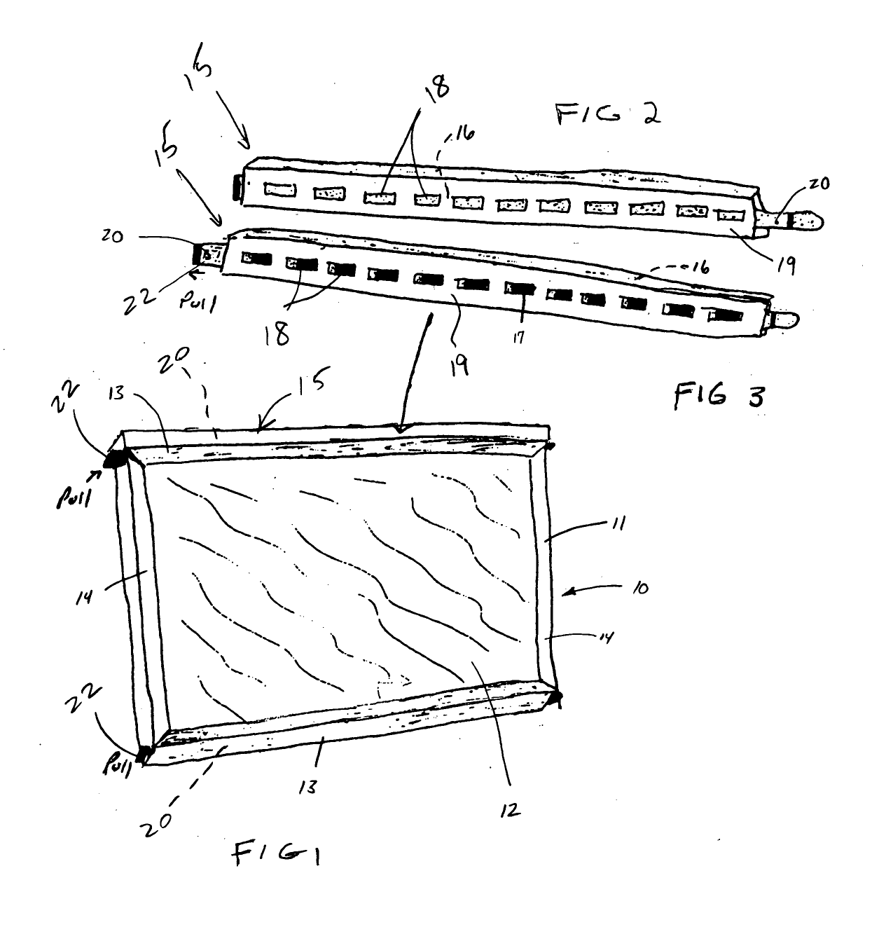

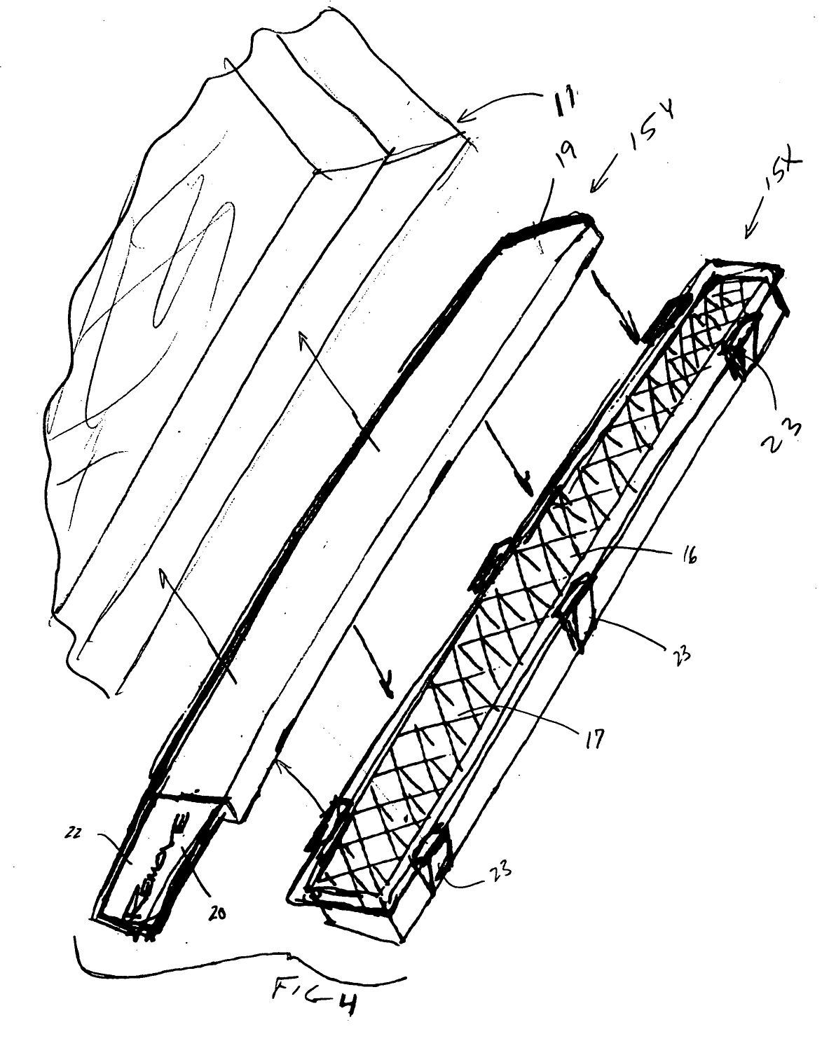

An air filter assembly 10 (FIG. 1-4) includes a perimeter frame 11 supporting an air filtering medium 12 defined by an opening within the perimeter frame 11. The illustrated perimeter frame 11 includes long and short side sections 13 and 14, respectively, forming a rectangle. A scent-holding member 15 is adheringly attached to each of the long sections 13. Each scent-holding member 15 defines an internal cavity 16 for holding scented material 17. Apertures or holes 18 are provided on an inside wall 19 of the perimeter frame 11 facing the air filtering medium 12. The apertures 18 align with side holes in the side sections 13 (and/or side sections 14), but are not directly subject to flow-through of air passing through the filtering medium 12 due to the lateral location of the member 15 on the frame 11. A semi-rigid cover or tape 20 slidably engages the side sections 13 and 14 (or slidably engages the member 15) and is movable to selectively partially (or fully) uncover the apertures 18, thus providing control of the scenting process and the concentration of scent in the air. By this arrangement, air passing through the filter assembly 10 is scented, but the scented material 17 is longer-lasting since it is drawn out of the internal cavity 16 rather than being blown out or evaporated by high air flow. In addition, the scenting process may be controlled by selectively withdrawing or positioning a pull tab 22 from the scent-holding member 15. Specifically, withdrawing the pull tab 22 selectively removes all or a portion of the cover or tape 20 to uncover apertures 18. Also, depending on the configuration, the scented material 17 can be replaced (see FIG. 4). Still further, the arrangement is attachable to a filter assembly of conventional construction, such as by adhesively attaching the scent-holding member 15 to side sections 13 or 14 by placing adhesive on an outer surface of wall 19 or the adjacent front and rear walls on the frame 11.

In particular, FIG. 4 depicts one method of constructing the scent-holding member 15 from a trough component 15X and a cover-like component 15Y. Snap assembly tabs 23 are formed on the side of trough component 15X to secure the cover component 15Y to the trough component 15X. Snap assembly brackets 23 are easily detachable, which makes refilling scented material 17 into the cavity 16 simple and fast. Upon refill, the cover or tape 20 is simply re-attached to the scent-holding member 15, and the scent-holding member 15 is subsequently re-attached to the perimeter frame 11. The tape 20 is slidably mounted on the cover-like component 15Y.

It is contemplated that the air filter assembly 10 including the scent-holding member 15 will have standard outer dimensions so that the filter assembly 10 will be useable in conventional residential forced air furnaces. As such, the frame 11 is slightly less in size than a conventional frame for an air filter for a residential forced air furnace. Nonetheless, it is conceived that the scent-holding member 15 may be constructed to be retrofitted to an existing filter frame assembly 10. Further, the scented material 17 disposed inside the cavity 16 of the scent-holding member 15 may include a variety of different scent-generating arrangements including a relatively solid block, a gel, a granular material, a powder, or an absorbent material saturated with a scented substance . . . or even a liquid.

In use, air passes through the filter medium 12 perpendicular to the planar extent of the filter frame assembly 10. A user may fully or partially withdraw the pull tab 22 from the perimeter frame 11 thereby removing all or a portion of the cover or tape 20 from the apertures 18. The scented material 17 disposed inside the cavity 16 is then exposed indirectly to air flow traveling through the filter frame assembly 10. The application of indirect air flow to the scented material 17 results in a relatively consistent volume of vaporized scented material 17 entering and dispersing with the air over the life of the filter frame assembly 10, since the scent is drawn slowly out of the cavity 16 via a “self-dispersion” vapor mechanism, rather than being forced out by heated air forced through the scent material medium. The concentration of emitted scent is easily controlled by a position of the cover 20 and the size of apertures 18.

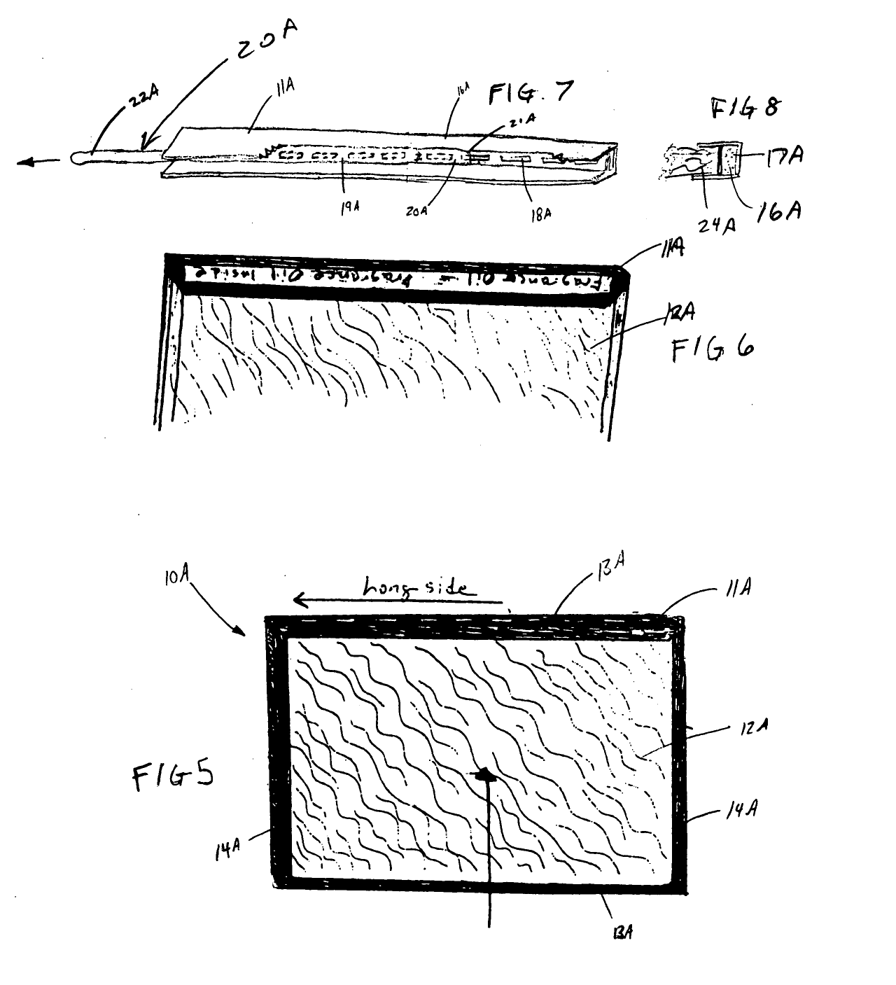

Additional modified versions of the air filter assembly 10 are illustrated in FIGS. 5-8, 9-12, and 13-16. In each version, identical and similar components and features are identified using the same number but with the addition of a letter “A”, “B” or “C”. This is done solely to reduce redundant discussion and not for another purpose.

The air filter assembly 10A (FIGS. 5-8) includes a perimeter frame 11A supporting an air filtering medium 12A, where the perimeter frame 11A includes long and short side sections 13A and 14A, respectively, that each define an internal cavity 16A holding scented material 17A. In assembly 10A, the features of scent-holding member 15 of FIG. 1 are incorporated into one or all of the side sections 13A-14A. Specifically, in FIG. 5, the side sections 13A and 14A are integrally formed as part of the perimeter frame 11A, and are not separate add-on components. Apertures or holes 18A are provided on an inside wall 19A of the perimeter frame 11A facing the air filtering medium 12A, with the apertures 18A not being directly subject to flow of air passing through the filtering medium 12A. A thin cover or tape 20A is positioned on the side sections 13A and 14A to selectively cover and uncover the apertures 18A. The cover or tape 20A is doubled up across the longitudinal extent of each side section 13A and 14A, forming a fold 21A at an opposite end from the pull tab 22A. Withdrawing the pull tab 22A selectively removes all or a portion of the cover or tape 20A. Removal of the tape or cover 20A allows air flow to pass by apertures 18A resulting in vapors 24A emanating from the internal cavity 16A.

The air filter assembly 10B (FIGS. 9-12) includes a perimeter frame 11B supporting an air filtering medium 12B, where the perimeter frame 11B includes long and short side sections 13B and 14B that each define an internal opening 19B′ for receiving a scent-holding member 15B within cavity 16B. As with previous embodiments, apertures or holes 18B are provided on an inside wall 19B of the perimeter frame 11B facing the air filtering medium 12B, with the apertures 18B not being directly subject to flow-through of air passing through the filtering medium 12B. Scented material 17B is carried by a T-shaped scent-holding member 15B that acts as a reservoir. The T-shaped scent-holding member 15B may be slid longitudinally into the cavity 16B through an open end 21B of the cavity 16B. A tape 20B is positioned on an inside surface of the T-shaped scent-holding member 15B. The tape 20B is selectively removed or peeled back to reveal apertures 18B having scented material 17B disposed therein prior to insertion of the T-shaped scent-holding member 15B into the cavity 16B. Alternatively, the tape 20B can be replaced with a rigid cover that slidably engages the scent-holding member 15B.

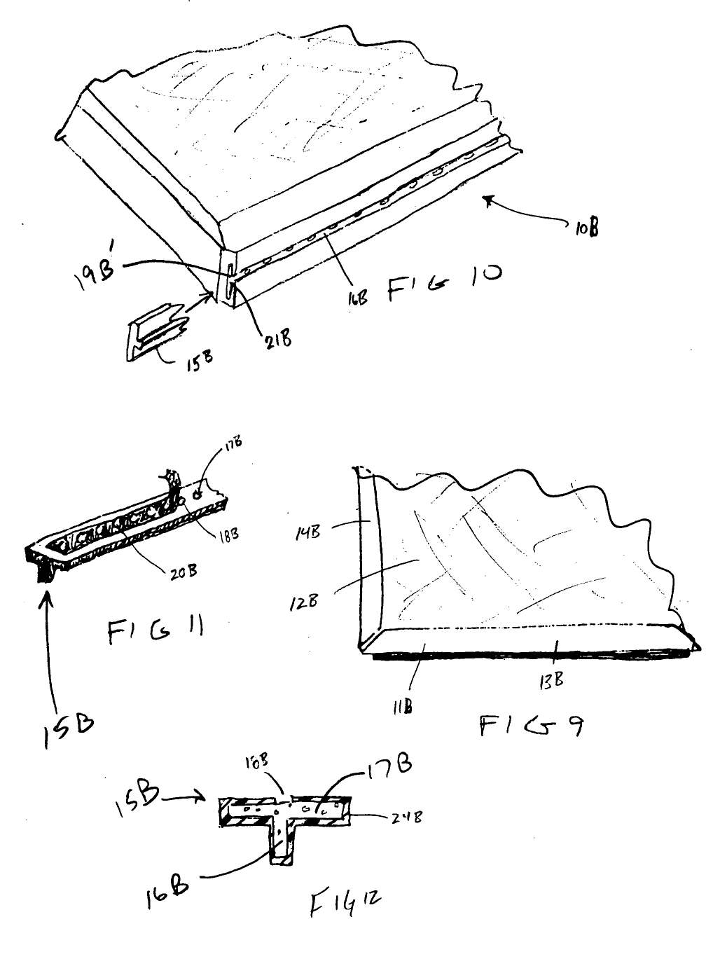

In assembly 10B (FIG. 10), the side sections 13B and 14B are shown as being integrally formed as part of the perimeter frame 11B, and are not separate add-on components. Nonetheless, it is to be understood that it could be. The illustrated arrangement permits T-shaped scent-holding member 15B containing the scented material 17B to be easily added to the perimeter frame 11B as needed without extraordinary measures.

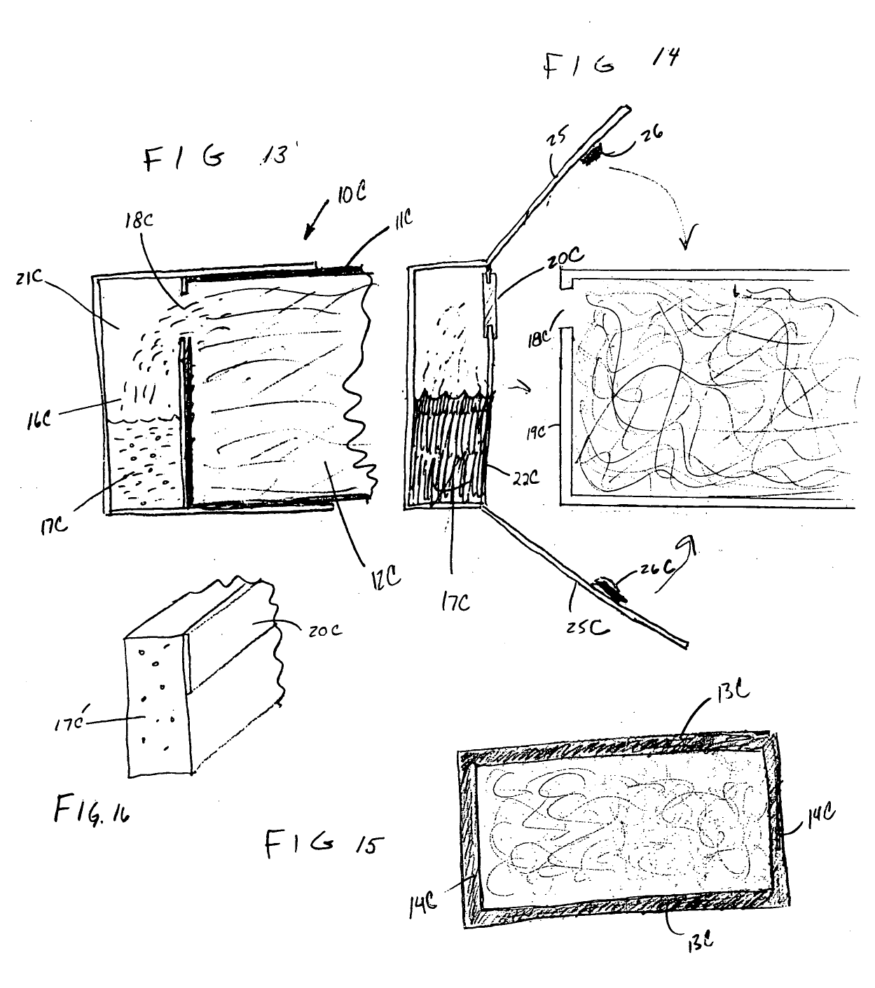

The air filter assembly 10C (FIGS. 13-16) is another version similar to the air filter assembly 10A (FIGS. 5-8), but the assembly 10C is modified to include a sliding cover 20C and further is modified to hold a scented material 17C that is liquid scent 17C (FIGS. 13-14) or a semi-fluid gel 17C′ (see FIG. 16). The perimeter frame 11C supports an air filtering medium 12C, with the perimeter frame 11C including long and short side sections 13C and 14C that each define an internal cavity 16C holding scented material 17C. The illustrated side sections 13C and 14C are integrally formed as part of the perimeter frame 11C and are not separate add-on components. Side sections 13C have flanges 25C that attach to the air filtering medium 12C at adhesive points 26C. Apertures or holes 18C are provided on an inside wall 19C of the perimeter frame 11C facing the air filtering medium 12C. The scented material 17C may be a liquid material that can be pumped into the cavity 16C by various means, such as through an open end 21C (or through a valve) into the cavity 16C or, as shown in FIG. 16, the scented material 17C may be a relatively solid block or semi-fluid gel that may be slid into the cavity 16C. The cover 20C slidably engages the inside wall 22C and selectively covers the apertures 18C. The cover 20C can be selectively slid along the wall 22C to selectively cover or reveal the scented material 17C.

It is to be understood that variations and modifications can be made on the aforementioned structure without departing from the concepts of the present invention, and further it is to be understood that such concepts are intended to be covered by the following claims unless these claims by their language expressly state otherwise.

1. An air filter assembly comprising:
a perimeter frame defining an opening adapted to permit flow of air being filtered, the perimeter frame including a scent-holding member defining a cavity having laterally-facing apertures that face the opening but that are located out of direct flow of the air;
a filtering medium adapted to filter the air and that is supported by the perimeter frame and positioned in the opening; and
a scented material located within the cavity of the scent-holding member for slow passage through the laterally-facing apertures into the flow of air.

2. The air filter assembly defined in claim 1, wherein the scent-holding member includes an elongated side section that forms an entire side of the perimeter frame.

3. The air filter assembly defined in claim 1, wherein the scent-holding member includes an elongated side section including first and second components that snap-attach together to form the cavity.

4. The air filter assembly defined in claim 1, wherein the perimeter frame includes a first side section and the scent-holding member is attached to the first side section.

5. The air filter assembly defined in claim 4, wherein the scent-holding member is adheringly attached to the first side section.

6. The air filter assembly defined in claim 4, wherein the scent-holding member includes an elongated first component attached to an outboard side of the first side section, and further includes a second component attached to the first component to form the cavity.

7. The air filter assembly defined in claim 1, wherein the scented material is non-liquid.

8. The air filter assembly defined in claim 7, wherein the scented material is a solid bar of material that slowly evaporates and that can be handled as a solid mass.

9. The air filter assembly defined in claim 1, wherein the scented material is configured to slide longitudinally into the cavity from an end of the side section.

10. The air filter assembly defined in claim 1, including a sliding cover engaging the scent-holding member and that is adapted to selectively cover and uncover the apertures.

11. The air filter assembly defined in claim 1, wherein a second scent-holding member is attached to a second side of the perimeter frame.

12. An air filter assembly comprising:
a perimeter frame defining an opening adapted to permit flow of air being filtered, the perimeter frame including a scent-holding member defining a longitudinally-extending cavity within the scent-holding member, the scent-holding member having apertures that face the opening but that are located out of direct flow of the air and further having an open end;
a filtering medium adapted to filter the air and that is supported by the perimeter frame and positioned in the opening; and
scented material adapted to pass through the open end into the cavity of the scent-holding member, and to be retained therein.

13. The air filter assembly defined in claim 12, wherein the scent-holding member forms an entire side of the perimeter frame.

14. The air filter assembly defined in claim 12, wherein the scent-holding member includes first and second components that snap-attach together to form the cavity.

15. The air filter assembly defined in claim 12, wherein the perimeter frame includes a first side section and the scent-holding member attaches to the first side section.

16. The air filter assembly defined in claim 12, wherein the perimeter frame includes a first side section and the scent-holding member adheringly attaches to the first side section.

17. An air filter assembly comprising:
a perimeter frame defining an opening adapted to permit flow of air being filtered and defining a cavity adjacent the opening and within the cavity, the perimeter frame defining a plurality of apertures facing the opening and including a strip of material that selectively covers the apertures, the strip being movable to uncover a desired number of the apertures;
a filtering medium adapted to filter the air and that is supported by the perimeter frame and positioned in the opening; and
a scented material in the cavity of a side section of the perimeter frame.

18. The air filter assembly defined in claim 17, wherein the strip forms a rigid sliding cover adapted to selectively cover the aperture.

19. The air filter assembly defined in claim 18, wherein the cover slidably engages the scent-holding member and comprises a rigid elongated tape.
